(19) United States
(12) Patent Application Publication
(10) Pub. No.: US 2026/0004875 A1
(43) Pub. Date: Jan. 1, 2026

(54) HOMOLOGOUS RECOMBINATION DEFICIENCY SCORING AND STATUS DETERMINATION

(71) Applicant: Integrated DNA Technologies, Inc., Coralville, IA (US)

(72) Inventors: Ryan Rogge, Erie, CO (US); Taylor R. Patterson, Longmont, CO (US); Allison Hadjis, Boulder, CO (US); Devin Tauber, Castle Rock, CO (US); Mark F. Rogers, Fort Collins, CO (US); Brent Lutz, Lafayette, CO (US); Laura Johnson, Boulder, CO (US); Trent K. Fridey, Cary, NC (US); David McConnell, Boulder, CO (US)

(21) Appl. No.: 19/253,188

(22) Filed: Jun. 27, 2025

Related U.S. Application Data
(60) Provisional application No. 63/665,621, filed on Jun. 28, 2024.

(51) Int. Cl. G16B 20/10; G16B 20/20

(52) U.S. Cl. CPC G16B 20/10; G16B 20/20

(57) ABSTRACT

A method of generating a homologous recombination deficiency score includes generating single nucleotide polymorphism (SNP) panel data describing allele abundance at each SNP locus of a plurality of SNP loci, generating double strand break (DSB) feature panel data describing nucleotide sequences at a plurality of DSB feature loci from the nucleic acid sample, generating allele specific copy number data for the plurality of SNP loci based on the SNP panel data, determining an entropy of the allele specific copy number data, comparing the DSB feature panel data to a known genomic sequence to identify a set of DSB mutations, determining a portion of the set of DSB mutations that are repaired by non-homologous end joining, and generating the homologous recombination deficiency score from the entropy of the allele specific copy number data and the portion of DSB mutations repaired by non-homologous end joining.

### CROSS-REFERENCE TO RELATED APPLICATION(S)

This application claims the benefit of U.S. Provisional Application No. 63/665,621, filed Jun. 28, 2024, and entitled “HOMOLOGOUS RECOMBINATION DEFICIENCY SCORING AND STATUS DETERMINATION,” the disclosure of which is hereby incorporated by reference in its entirety.

### FIELD OF THE INVENTION

The present disclosure relates to homologous repair deficiency and, more particularly, systems and methods for determining homologous repair deficiency status.

### BACKGROUND

The homologous recombination repair (HRR) pathway is one pathway utilized by cells to repair double strand breaks (DSBs). The HRR pathway is associated with fewer mutations than other pathways used by cells to repair DSBs, such as non-homologous end joining (NHEJ), and defects to the HRR pathway can result in genomes having a relatively high accumulation of mutations due to errors during DSB repair.

Homologous recombination deficiency (HRD) is a phenotype associated with significant disruptions to the HRR pathway and, in some instances, an inability to repair the genome using homologous recombination. There are multiple mutant genotypes associated with the HRD phenotype. However, it is possible to classify whether a cell has an HRD phenotype by detecting “genomic scarring” or “genomic instability,” which refer to particular patterns of mutations that often occur in HRD phenotypes. Notably, tumors that exhibit HRD are associated with sensitivities to particular classes of drugs, such as a poly(adenosine diphosphate ribose) polymerase (PARP) inhibitors and other classes of drugs capable of inhibiting proteins involved in DNA repair.

### SUMMARY

An example of a method of generating a homologous recombination deficiency score includes generating single nucleotide polymorphism (SNP) panel data from a nucleic acid sample and generating double strand break (DSB) feature panel data from the nucleic acid sample. The SNP panel data describes allele abundance at each SNP locus of a plurality of SNP loci and the DSB feature panel data describes nucleotide sequences at a plurality of DSB feature loci. Each DSB feature locus of the plurality of DSB feature loci includes at least one sequence feature associated with DSBs. The example of the method further includes generating allele specific copy number data for the plurality of SNP loci based on the SNP panel data and determining an entropy of the allele specific copy number data, as well as comparing the DSB feature panel data to a known genomic sequence to identify a set of DSB mutations and determining a portion of the set of DSB mutations that are repaired by non-homologous end joining. The example of the method yet further includes generating the homologous recombination deficiency score from the entropy of the allele specific copy number data and the portion of DSB mutations repaired by non-homologous end joining.

An example of a system for generating homologous recombination deficiency scores includes a processor and at least one memory encoded with instructions. The instructions, when executed, cause the processor to receive SNP panel data for a nucleic acid sample and also receive DSB feature panel data for the nucleic acid sample. The SNP panel data describes allele abundance at each SNP locus of a plurality of SNP loci and the DSB feature panel data describes nucleotide sequences at a plurality of DSB feature loci. Each DSB feature locus of the plurality of DSB feature loci includes at least one sequence feature associated with DSBs. The instructions, when executed, further cause the processor to generate allele specific copy number data for the plurality of SNP loci based on the SNP panel data and determine an entropy of the allele specific copy number data, as well as receive known genomic sequence data for a known genomic sequence, compare the DSB feature panel data to the known genomic sequence to identify a set of DSB mutations, and determine a portion of the set of DSB mutations that are repaired by non-homologous end joining. The instructions, when executed, further cause the processor to generate a homologous recombination deficiency score from the entropy of the allele specific copy number data and the portion of insertion-deletion mutations repaired by non-homologous end joining.

An example of a method of determining whether a tissue sample has a homologous repair deficiency phenotype includes generating SNP panel data describing allele abundance at each SNP locus of a plurality of SNP loci, generating allele specific copy number data for the plurality of SNP loci based on the SNP panel data, determining an entropy of the allele specific copy number data, and determining whether the entropy is above threshold entropy indicative of whether the tissue sample has the homologous repair deficiency phenotype. The SNP panel data is generated from a nucleic acid sample derived from the tissue sample.

An example of system for determining whether a tissue sample has a homologous repair deficiency phenotype includes a processor and at least one memory encoded with instructions. The instructions, when executed, cause the processor to receive SNP panel data describing allele abundance at each SNP locus of a plurality of SNP loci, generate allele specific copy number data for the plurality of SNP loci based on the SNP panel data, determine an entropy of the allele specific copy number data, and determine whether the entropy is above threshold entropy indicative of whether the tissue sample has a homologous repair deficiency phenotype. The SNP panel data is generated from a nucleic acid sample derived from the tissue sample.

A further example of a method of determining whether a tissue sample has a homologous repair deficiency phenotype includes generating DSB feature panel data describing nucleotide sequences at a plurality of DSB feature loci, comparing the DSB feature panel data to a known genomic sequence to identify a set of DSB mutations, determining a portion of the set of DSB mutations that are repaired by non-homologous end joining, and determining whether the portion of the set of DSB mutations that are repaired by non-homologous end joining is above a threshold portion of DSB mutations repaired by non-homologous end joining indicative of whether the tissue sample has the homologous repair deficiency phenotype. Each DSB feature locus of the plurality of DSB feature loci includes at least one sequence feature associated with DSBs and the DSB feature panel data is generated from a nucleic acid sample derived from the tissue sample.

A further example of a system for determining whether a tissue sample has a homologous repair deficiency phenotype includes a processor and at least one memory encoded with instructions. The instructions, when executed, cause the processor to receive DSB feature panel data describing nucleotide sequences at a plurality of DSB feature loci, receive known genomic sequence data for a known genomic sequence, compare the DSB feature panel data to the known genomic sequence to identify a set of DSB mutations, determine a portion of the set of DSB mutations that are repaired by non-homologous end joining, and determine whether the portion of the set of DSB mutations that are repaired by non-homologous end joining is above a threshold portion of DSB mutations repaired by non-homologous end joining indicative of whether the tissue sample has the homologous repair deficiency phenotype. Each DSB feature locus of the plurality of DSB feature loci includes at least one sequence feature associated with DSBs and the DSB feature panel data is generated from a nucleic acid sample derived from the tissue sample.

The present summary is provided only by way of example, and not limitation. Other aspects of the present disclosure will be appreciated in view of the entirety of the present disclosure, including the entire text, claims, and accompanying figures.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a flow diagram of an example of a method of calculating entropy of chromosomal allele-specific copy number data.

FIG. 2 is an example of a combined plot of absolute copy number, relative copy number, and allele distribution for an HRD positive tumor sample derived from single nucleotide polymorphism panel data.

FIG. 3 is a schematic diagram of an example of absolute copy number data similar to the data plotted in FIG. 2 and depicting data used to generate probabilities for entropy calculation.

FIG. 4 is a flow diagram of a method of identifying a portion of insertion-deletion mutations repaired by non-homologous end joining.

FIG. 5 is a flow diagram of an example of a method of scoring and, optionally, classifying homologous recombination repair.

FIG. 6 is a schematic diagram of a system for performing any of the methods of FIG. 1, FIG. 4, and FIG. 5.

FIG. 7 is a plot of entropy calculated according to the method of FIG. 1 against genomic instability score for a cohort of thirty-two samples.

FIG. 8 is a plot of portion of insertion-deletion mutations repaired by non-homologous end joining, calculated according to the method of FIG. 4, against genomic instability score for the cohort of thirty-two samples depicted in FIG. 7.

FIG. 9A is a plot of homologous recombination deficiency score generated according to the method of FIG. 5 against genomic instability score for the cohort of thirty-two samples depicted in FIGS. 7-8.

FIG. 9B is the plot shown in FIG. 9A also depicting thresholds usable for homologous recombination deficiency classification.

FIG. 10 is a combined frequency graph of the homologous recombination deficiency score data shown in FIGS. 9A-9B.

While the above-identified figures set forth one or more examples of the present disclosure, other examples are also contemplated, as noted in the discussion. In all cases, this disclosure presents the invention by way of representation and not limitation. It should be understood that numerous other modifications and examples can be devised by those skilled in the art, which fall within the scope and spirit of the principles of the invention. The figures may not be drawn to scale, and applications and examples of the present invention may include features and components not specifically shown in the drawings.

### DETAILED DESCRIPTION

The present disclosure relates to systems and methods for generating information entropies of allele-specific copy number (ASCN) data, generating values descriptive of non-homologous end joining (NHEJ) utilization for repairing double strand breaks (DSBs), and further for generating homologous repair deficiency (HRD) scores. More generally, the present disclosure relates to systems and methods for determining whether tissue samples are HRD positive (i.e., have an HRD phenotype). Tumors having HRD positive phenotypes are sensitive to various treatment methods, such as a poly(adenosine diphosphate ribose) polymerase (PARP) inhibitors. The entropy and NHEJ characterizations disclosed herein provide values that correlate to genomic instability score (GIS), an established method of characterizing HRD status. As such, the entropy and NHEJ characterizations disclosed herein are also able to be used to determine HRD status. Further, the present disclosure also provides systems and methods for generating a combined HRD score that incorporates both genomic information entropy and NHEJ repair utilization into a single HRD score with higher correlation to GIS than either entropy or NHEJ repair utilization alone.

Notably, existing methods characterizing HRD status generally rely on large sequencing volumes to detect widespread genomic scarring and often measure genomic scarring via a combination of loss of heterozygosity (LOH), large-scale transitions (LSTs), and telomeric allelic imbalance (TAI). Some methods of quantifying genomic scarring also incorporate other descriptors of genomic instability, such as copy number variation, breakpoint quantity, etc. As such, existing methods often require large panel sizes (i.e., and thus large numbers of primers or probes for enrichment) and a significant number of sequencing reads. HRDetect, for example, requires whole genome sequencing to make HRD determinations (Davies H, Glodzik D, Morganella S, Yates L R, Staaf J, Zou X, Ramakrishna M, Martin S, Boyault S, Sieuwerts A M, Simpson P T, King T A, Raine K, Eyfjord J E, Kong G, Borg Å, Birney E, Stunnenberg H G, van de Vijver M J, Børresen-Dale A L, Martens J W, Span P N, Lakhani S R, Vincent-Salomon A, Sotiriou C, Tutt A, Thompson A M, Van Laere S, Richardson A L, Viari A, Campbell P J, Stratton M R, Nik-Zainal S. HRDetect is a predictor of BRCA1 and BRCA2 deficiency based on mutational signatures. Nat Med. 2017 April; 23(4):517-525. doi: 10.1038/nm.4292). As additional examples, the TruSight Oncology 500 HRD panel includes coverage of 25,000 SNPs (Illumina); the Northeastern German Society for Gynecologic Oncology (NOGGO) GIS assay targets 20,000 SNPs; and the AmoyDx HRD Focus Panel targets 24,000 SNPs. Conversely, the genomic information entropy quantification described herein can be performed with only 5,000 primers targeting 5,000 SNP sites and the NHEJ repair characterization method described herein can be performed with only 130 primers. Further, these existing HRD characterization methods often require tens of millions of reads per library. A combined HRD score according to the present disclosure can be generated with, for example, only 12 million reads per library.

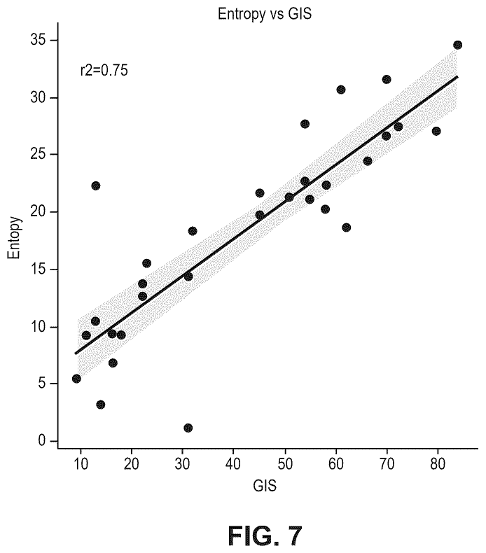

As such, the systems and methods of the present disclosure significantly reduce the quantity of materials and the number of sequencing reads required to characterize HRD status, and thereby significantly reduce the cost associated with HRD characterization. Further, as will be shown with respect to the examples described subsequently and the discussion of FIGS. 7-10, the indicators of HRD described herein correlate strongly with GIS and, accordingly, are able to accurately identify HRD status using a reduced number of genome targets and a reduced number of sequencing reads as compared to conventional genomic scarring methods, such as GIS.

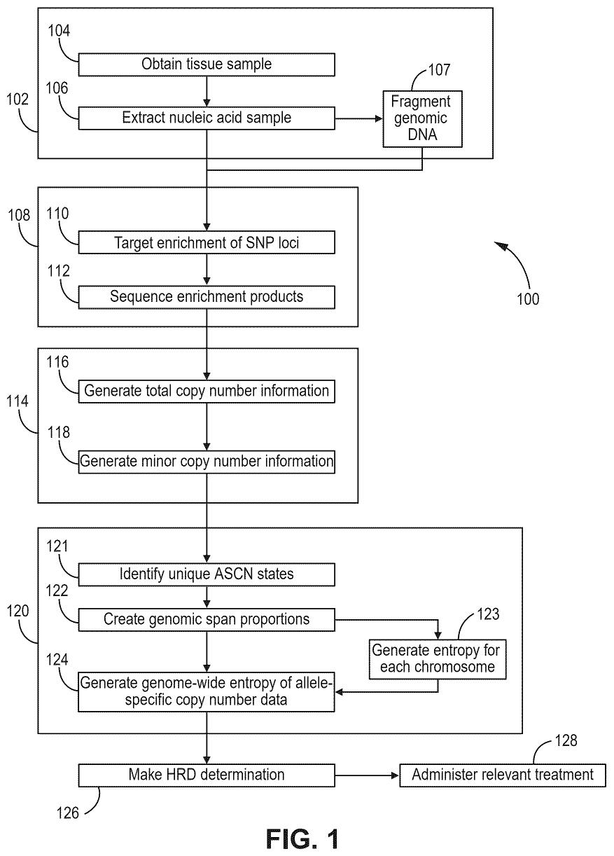

FIG. 1 is a flow diagram of method 100, which is a method of assessing genomic entropy. Method 100 includes steps 102-128 of preparing a nucleic acid sample (step 102), obtaining a tissue sample (step 104), extracting a nucleic acid sample (step 106), fragmenting genomic DNA (step 107), generating SNP panel data (step 108), performing target enrichment of SNP loci (step 110), sequencing the enrichment products (step 112), generating ASCN data (step 114), generating total copy number information (step 116), generating minor copy number information (step 118), generating an entropy the ASCN data (step 120), identifying unique ASCN states (step 121), creating genomic span proportions (step 122), generating entropy for each chromosome (step 123), generating genome-wide entropy of the ASCN data (step 124), making an HRD determination (step 126), and administering a relevant treatment (step 128). As is depicted in FIG. 1, steps 104-107 are substeps of step 102, steps 110-112 are substeps of step 108, steps 116-118 are substeps of step 114, and steps 122-124 are substeps of step 120.

In step 102, a nucleic acid sample is prepared. The nucleic acid sample is suitable for amplification and sequencing and, in some examples, can be produced via steps 104-107.

In step 104, a tissue sample is obtained. The tissue sample can be obtained by a human operator and can be of any suitable tissue. In at least some examples, the tissue sample can be or include tissue extracted from a tumor (e.g., via a biopsy).

In step 106, a nucleic acid sample is extracted from the tissue sample obtained in step 104. The nucleic acid sample can be, for example, genomic DNA, RNA, ctDNA, any other nucleic acid polymer, or mixture of any of the foregoing. Cells of the tissue sample can be lysed and the soluble nucleic acid-containing fraction can be separated from the insoluble fraction containing cellular debris. Cells of the tissue sample can be lysed by any suitable technique such as, for example, via a detergent and the soluble and insoluble fractions can be separated by any suitable method, such as by high-speed centrifugation. In some examples, the nucleic acid sample can be precipitated (e.g., via one or more organic solvents) and resuspended prior to use in subsequent steps of method 100. More generally, step 106 can be performed according to any known technique for isolating a nucleic acid sample from a tissue sample. Where the nucleic acid sample is genomic DNA, the nucleic acid sample can include all genomic DNA or less than all genomic DNA. In some examples, the nucleic acid sample extracted in step 106 includes at least all autosomal DNA of a tumor cell.

In step 107, genomic DNA is fragmented. Step 107 is an optional step of method 100 and can be performed in examples of method 100 in which the nucleic acid sample isolated in step 106 is or contains genomic DNA, and further in examples where it is desirable to shear that genomic DNA prior to target enrichment (e.g., in hybridization capture workflows). The genomic DNA can be fragmented via any suitable known shearing technique, including any suitable physical shearing technique (e.g., sonication) and/or any suitable enzymatic DNA fragmentation technique, among other options.

Following step 106 or, in relevant examples, step 107, method 100 proceeds to step 108. In step 108, SNP panel data is generated. The SNP panel data in step 108 provides information related to the base identity at various SNP loci within the nucleic acid sample and, further, information describing the abundance of each base identity or each SNP-containing gene. The abundance of each base identity can be, for example, the abundance of nucleotide fragments containing each base identity. In particular, the SNP panel data generated in step 108 provides base identity at SNP loci, which allows the SNP data generated in step 108 to be used to generate allele specific copy number data in subsequent step 114. In some examples, step 108 can be performed using a next-generation sequencing technique. In other examples, any other suitable technique for genotyping SNPs can be performed in step 108.

In step 110, target enrichment of SNP loci to be characterized to generate ASCN data is performed. Target enrichment can be performed by, for example, amplification using primers targeting sites near to and/or adjacent to SNP loci. The primers can be, e.g., gene-specific primers for genes containing SNPs. Target enrichment in step 110 is performed via any suitable polymerase chain reaction (PCR) amplification technique and, in at least some examples, is performed using anchored multiplex PCR (Archer; also described by Zheng, Z., Liebers, M., Zhelyazkova, B. et al. Anchored multiplex PCR for targeted next-generation sequencing. Nat Med 20, 1479-1484 (2014) https://doi.org/10.1038/nm.3729). The SNP loci intended to be sequenced in subsequent step 112 can be targeted via gene-specific primers using AMP. AMP can be performed according to known techniques and optionally using two gene-specific primers for each SNP loci to increase amplicon specificity.

In other examples, target enrichment can be performed by hybridization capture of fragmented DNA. Sequencing adapters then be ligated to fragmented DNA to generate a genomic library. The genomic library can be denatured and, subsequently, probes targeting sequences adjacent to and/or including SNP locations are hybridized to the target DNA sequences within the genomic library. The probes can be of any suitable length and can target any suitable number of regions within a particular DNA molecule (e.g., a molecular inversion probe targeting two regions of a target DNA molecule). The probes can be, for example, immobilized (e.g., attached to a suitable solid surface). Additionally and/or alternatively, the probes can be suspended in a solution and can be captures using, e.g., magnetic beads having functional groups configured to interact with the probes. In these examples, one or more additional PCR reactions can be optionally performed to amplify target DNA regions prior to sequencing.

Step 110 can be performed for any suitable number of SNP loci but, in at least some examples, step 110 is performed for approximately 5,000 SNP loci. In at least some examples, SNP loci targeted in step 110 are only loci of autosomal chromosomes. The SNP loci targeted in step 110 are generally SNP loci that are putatively or likely to be heterozygous, but not all SNP loci enriched in step 110 and sequenced in step 112 may be heterozygous. Various steps required for library preparation and other steps necessary prior to sequencing can be performed in step 110.

In step 112, the enriched products generated in step 110 are sequenced. The enriched products can be, for example, amplicons in examples where PCR was used to amplify target sequences. The enriched products can also be, for example, adapted genomic DNA fragments (i.e., having ligated sequencing adapters) obtained in step 110. The enriched nucleotides can be sequenced using any suitable sequencing technique to provide base identity information for the SNP loci targeted in step 110. Sequencing can be performed by any suitable sequencing technique, but in at least some examples is performed using a sequencing-by-synthesis technique. The sequencing-by-synthesis technique can be, for example, Illumina dye sequencing (Illumina), ion semiconductor sequencing (e.g., Thermo Fisher Ion Torrent sequencing), or any other suitable sequencing-by-synthesis technique. The sequencing data generated in step 112 provides both identity and count information for each SNP allele.

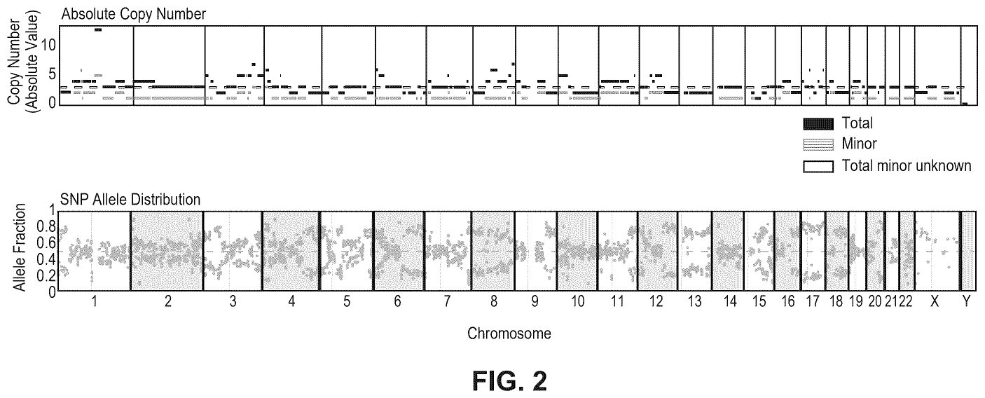

In step 114, ASCN data is generated based on the SNP panel data generated in step 106. The ASCN data generated in step 114 FIG. 2 is one example of ASCN data that can be generated in step 114 based on SNP panel data (i.e., SNP genotyping data) that is generated in prior step 108. Specifically, FIG. 2 is a combined plot of absolute copy number and allele distribution for an HRD positive tumor sample derived from single nucleotide polymorphism data. Genomic DNA from HRD positive ovarian tissue was amplified by AMP with a panel of primers targeting various known SNP sites. FIG. 2 depicts absolute copy number for various SNP-containing genes as well as SNP allele distribution. Notably, data of the type shown in FIG. 2, or equivalent data, can also be generated using any other suitable target enrichment method.

Both plots in FIG. 2 share a common x-dimension, which defines chromosomal location of each SNP locus examined as well as genomic span (in base pairs) of each SNP-containing allele. Notably, although the x-dimension in FIG. 2 includes chromosomal locations on the Y chromosome, SNP data collected to create the ASCN information in FIG. 2 does not include SNP sites located on the Y chromosome (i.e., due to the tissue being derived from ovarian tissue). The y-dimension of the plot of SNP allele distribution defines the relative proportion of each possible identity at each SNP site as determined by the SNP panel data generated according to step 108. The SNP allele distribution information can be used to determine absolute copy number information also shown in FIG. 2. Generation of absolute copy number information can be performed using any suitable known algorithm for calling absolute copy number from SNP panel data, such as allele-specific copy number analysis of tumors (ASCAT), fraction and allele-specific copy number estimates from tumor sequencing (FACETS), ABSOLUTE, PureCN, etc. The y-dimension of the absolute copy number data is copy number value and the plot of absolute copy number includes calls of total copy number and minor copy number (including total copy number for alleles for which minor copy number is not called).

Returning to method 100 and FIG. 1, in all examples of step 114, information descriptive of the relative frequency of the minor allele is generated for at least some genes having SNP loci interrogated by the SNP panel in step 108. In the depicted example, relative frequency information for the minor allele for a given gene can be used to generate the absolute copy number for all alleles (i.e., total copy number). In the example of method 100 depicted in FIG. 1, step 114 can optionally be performed by generating total copy number information in step 116 and generating minor copy number information in step 118.

The total copy number information generated in step 116 and the minor copy number information generated in step 118 can be called using any suitable method for calling copy number based on SNP panel data, as described previously, and in some examples can be generated according to the workflow outlined above with respect to FIG. 2. As referred to herein, “minor copy number” and “total copy number” information refers to copy number of SNP alleles. Further, as referred to herein, characterization of copy number for “all” genes containing SNP loci targeted in step 108 refers to the characterization of all genes targeted in step 108 for which the relative frequency of a minor allele can practically be called, including examples (e.g., the example of FIG. 2) in which minor allele frequency cannot be accurately called for a subset of genes containing SNP loci targeted in step 108.

In step 120, the entropy of the ASCN data generated in step 114 is determined. As referred to herein, “entropy” of ASCN data refers to the Shannon or information entropy of that data. Entropy of ASCN data as calculated according to method 100 is correlated with the number of DSB events that have been repaired in a given genome. As such, entropy of ASCN data increases as genomic instability increases, and entropy of ASCN data can be used to approximate GIS.

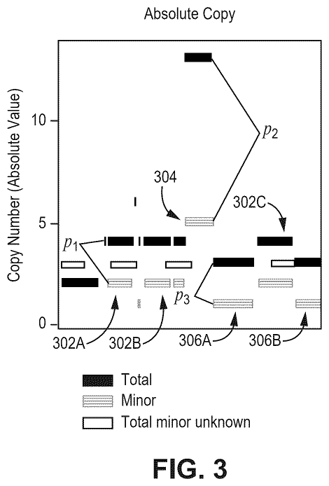

Steps 121-124 outline one example by which entropy of ASCN data can be generated and, in other examples, entropy of ASCN data can be calculated using any suitable method. In step 121, unique allele-specific copy number states are identified based on the ASCN data generated in step 114. Unique copy number states are identified based on the total copy number information generated in step 116 and the minor copy number information generated in step 118. Unique combinations of total copy number and minor copy number form each unique copy number state and, in some examples, multiple alleles may have the same copy number state. FIG. 3 is a schematic diagram of a subset of absolute copy number data and illustrates several copy number states. FIG. 3 depicts unique copy number states p₁, p₂, and p₃, which are copy number states of alleles 302A-C, 304, and 306A-B, respectively. The data in FIG. 3 is taken from the data for chromosome 1 of the ASCN data depicted in FIG. 2.

In step 122, genomic span proportions are created for each unique copy number state identified in step 122. Proportion information can be determined by, for each copy number state, determining the genomic span belonging to the alleles having the copy number state as well as the overall genomic span belonging to all copy number states. The ratio of the genomic span for a particular copy number instance to the overall genomic span of all copy number states is one proportion used in the entropy calculation in subsequent steps 123 and/or 124. Genomic span can be measured in, for example, base pairs or any other suitable unit. Where more than one allele has the same unique ASCN state (i.e., the same minor and total copy numbers), the genomic span of all alleles having that ASCN state is used to calculate the genomic span proportion for that ASCN state.

In some examples, proportions calculated in step 122 can be calculated in a chromosome-specific manner (e.g., in examples of method 100 including step 123). That is, step 124 can be performed by, for each unique copy number state within a given chromosome, generating the ratio of the genomic span of that unique copy number state within that chromosome to the genomic span of all copy number states within that chromosome. Additionally and/or alternatively, proportions generated in step 122 can be generated in a genome-wide manner. That is, for each unique copy number state within an entire genome, a proportion can be calculated by generating the ratio of the genomic span of that unique copy number state within the entire genome to the total genomic span of all observed unique copy number states. Other options for calculating genomic span proportions are possible, and the aforementioned examples are two illustrative options.

After step 122, method 100 optionally proceeds to step 123 or step 124. Step 123 is included in examples where it is advantageous to exclude the effects on ASCN from various conditions that may affect or skew a whole-genome entropy calculation. For example, changes to copy number present in the ratios generated in step 122 may result in part from whole chromosome aneuploidy. However, changes to copy number from whole chromosome aneuploidy (i.e., resulting in copy number changes across an entire chromosome) and other changes to copy number that arise from improper chromosome segregation during cell division do not result from DSB repair, but could affect entropies calculated directly from genome-wide copy number information. Other conditions or circumstances may result in count number changes unrelated to DSB repair and the aforementioned example of whole chromosome aneuploidy is merely one exemplary cause of alterations to copy number that is unrelated to DSB repair. Advantageously, inclusion of step 123 causes method 100 to perform a per-chromosome entropy calculation that reduces skew to entropy calculations imparted by copy number mutations that are unrelated to DSB repair. Per-chromosome entropy calculated in step 123 can then be aggregated (e.g., by averaging or by summation) in subsequent step 124. Step 123 can be performed in examples where genomic span proportions were generated in a chromosome-specific manner in step 122.

In step 124, a genome-wide entropy is calculated. In examples of method 100 omitting step 123, genome-wide entropy can be calculated based on all ratios calculated in step 122. In examples of method 100 including method 123, entropy can be calculated in step 124 by aggregating the chromosome-specific entropies generated in step 123. The chromosome-specific entropies can be aggregated by, e.g., averaging or summation.

Each entropy calculated in step 123 and/or step 124 can be calculated, for example, according to the following Equation 1:

$${H}^{′} = - {\sum }_{i = 1}^{R} {p}_{i} {log}_{2} {p}_{i} \qquad [ Equation 1 ]$$

where H′ is entropy, pᵢ is a single proportion of the proportions generated in step 122 (i.e., one of the genomic span proportions), and R is the number of proportions being summed. In step 123, R is the number of unique copy number states for which proportions were generated in step 122 and which belong to the chromosome for which entropy is being calculated. In step 124, R is the total number of unique copy number states generated in step 122. While information entropy generally is calculated using probabilities, the genomic span proportions for unique allele-specific copy number states created in step 122 can be used in substantially the same manner as probabilities to generate information entropy. H′ in Equation 1 has units of shannons (Sh) or bits. In other examples, Equation 1 can include a logarithm function with a different base (i.e., other than logarithm base 2) and H′ can consequently have different units, and other steps, formula, and equations disclosed herein that are dependent an H′ value produced according to Equation 1 can be adjusted accordingly.

In step 126, an HRD determination is made based on the entropy generated in step 120. HRD status can be determined using a threshold entropy associated with HRD positive and/or HRD negative phenotypes. Step 126 is an optional step of method 100 and is performed in examples where it is desirable to determine HRD status for the tissue sample obtained in step 102, such as in examples where the tissue sample is a tumor sample. The threshold can be determined by, for example, correlating entropy generated according to method 100 with GIS or another known standard for characterizing HRD status. Steps 102-120 (including any relevant substeps) can be performed for cell lines for which GIS is known or, alternatively, can be performed for cell lines for which GIS can be calculated. GIS can be calculated using an existing technique, such as the LOH+LST+TAI described by Patel et. al (Patel J N, Braicu I, Timms K M, Solimeno C, Tshiaba P, Reid J, Ganapathi R N. Characterisation of homologous recombination deficiency in paired primary and recurrent high-grade serous ovarian cancer. Br J Cancer. 2018; 9:1060-1066. doi: 10.1038/s41416-018-0268-6). Entropy can be plotted against GIS and a linear regression can be used to generate a correlative equation between GIS and genome-wide entropy. A threshold GIS value can then correlated to a corresponding entropy using the regression model and the resultant entropy threshold can be used in step 126.

In step 128, a treatment suitable for targeting HRD positive tumor cells is administered to a patient from which the tissue sample was obtained in step 102. Step 128 is an optional step of method 100 and is performed in examples in which the tissue sample is a tumor sample and in which it is desirable to administer a treatment to a patient based on the HRD status determination made in step 126. In examples of method 100 including step 128, step 128 only performed in examples of method 100 in which a tissue sample was categorized as HRD positive in step 126, such that performance of step 128 is conditional on the determination made in step 126. The treatment administered can be, for example, PARP inhibitor known to be effective in treating tumors deficient in homologous recombination (e.g., BRCA and BRCA-like tumors). PARP inhibitors are only one exemplary class of treatment suitable for treating HRD positive tumors, and other suitable treatments can be used in step 128.

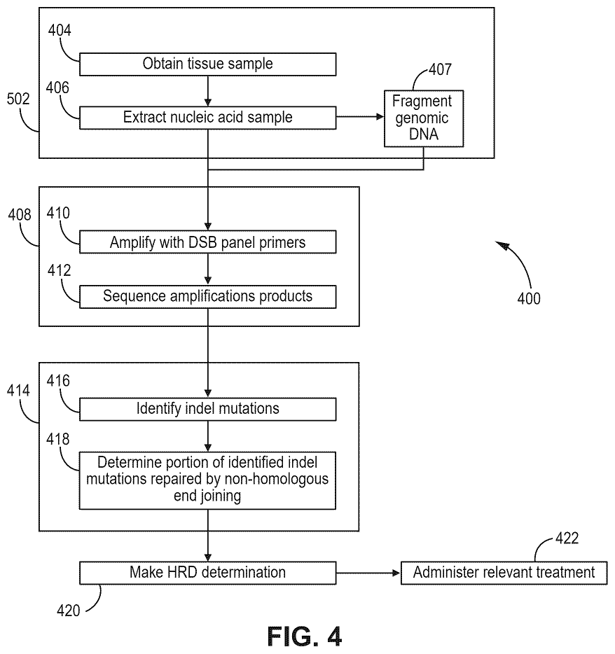

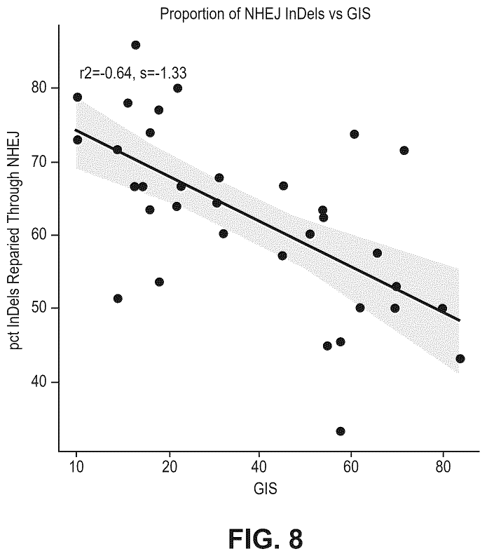

FIG. 4 is a flow diagram of method 400, which is a method of quantifying cellular NHEJ utilization. Method 400 characterizes regions that are likely to be susceptible to DSBs. In HRD positive cells, the proportion of DSBs repaired by NHEJ is altered as compared to HRD negative cells, as HRD positive cells lack robust homologous repair and instead rely on different repair pathways to correct DSBs. In particular, and as is shown in FIG. 8, discussed subsequently, the data disclosed herein evidences a negative correlation between NHEJ repair activity and HRD status.

Method 400 includes steps 402-422 of preparing a nucleic acid sample (step 402), obtaining a tissue sample (step 404), extracting a nucleic acid sample (step 406), fragmenting genomic DNA (step 407), generating DSB panel data (step 408), amplifying the nucleic acid sample with DSB panel primers (step 410), sequencing amplification products (step 412), determining a portion of DSBs repaired by NHEJ (step 414), identifying indel mutations based on the sequencing data (step 416), determining a portion of the identified indel mutations repaired by NHEJ (step 418), making an HRD determination (step 420), and administering a relevant treatment (step 422). As is depicted in FIG. 4, steps 404-407 are substeps of step 402, steps 410-412 are substeps of step 408, and steps 416-418 are substeps of step 414.

Steps 402-407 are substantially similar to steps 102-107 of method 100 (FIG. 1), respectively, and the discussion of steps 102-107 is applicable to steps 402-407 of method 400 (FIG. 4), respectively. In step 408, DSB panel data is generated. Step 408 is substantially similar to step 410, but provides nucleic acid sequence at regions where DSBs are likely to have occurred. Step 408 can include a target enrichment step that targets genome regions that include at least one feature associated with double strand breaks. Inverted repeat sequences are one type of DNA feature that is prone to DSBs. Inverted repeats are susceptible to forming cruciform structures that result in DSBs (Lu S, Wang G, Bacolla A, Zhao J, Spitser S, Vasquez K M. Short Inverted Repeats Are Hotspots for Genetic Instability: Relevance to Cancer Genomes. Cell Rep. 2015 Mar. 17; 10(10):1674-1680. doi: 10.1016/j.celrep.2015.02.039) and, consequently, are often sites of insertion and deletion mutations. Inverted repeats are targeted by optional step 410, as described subsequently, but in other examples, other types of features that are prone to DSBs can be targeted in substantially the same manner as described in the discussion of steps 410-412 herein. Other steps of method 400 can be similarly adapted to identify loci of mutations resulting from those DSB-prone DNA features.

Step 408 can optionally be performed via steps 410-412. In step 410, the nucleic acid sample isolated in step 406 (and optionally fragmented in step 407) is amplified with inverted repeat panel primers. Step 410 is substantially similar to step 110, but is performed with primers (i.e., for amplification-based enrichment) and/or probes (i.e., for hybridization-based enrichment) targeting inverted repeat sites within the genome. The primers and/or probes used in step 410 can, for example, target (i.e., via homology) inverted repeat sequences known or suspected to occur within the genome of the tissue sample obtained in step 404.

In step 412, the amplificons generated in step 410 are sequenced. Step 412 can be performed in the same or substantially the same manner as described with respect to step 112 of method 100 (FIG. 1), and the description herein of step 112 is applicable to step 412.

In other examples, other sequences where indels and/or other mutations resulting from DSB events can be targeted using different primers and/or probes in step 410, but steps 410-412 can be performed in substantially the same manner to target those other sequences. For example, sequences that give rise to DNA molecules or regions having unusual secondary structure can be analyzed in step 408 (and in adaptations of steps 410-412), such as poly dA:dT repeats, G-Quadruplexes, and R-loops. Additionally and/or alternatively, genomic regions empirically associated with DSB occurrence can be analyzed in step 408 (and in adaptations of steps 410-412), such as common fragile sites (CFS; Fungtammasan A, Walsh E, Chiaromonte F, Eckert K A, Makova K D. A genome-wide analysis of common fragile sites: what features determine chromosomal instability in the human genome? Genome Res. 2012 June; 22(6):993-1005. doi: 10.1101/gr.134395.111).

In step 414, the sequence information generated in step 408 (e.g., via steps 410-412) is analyzed to determine a portion of DSBs that were repaired by NHEJ. In some examples, step 414 can be performed via steps 416-418. In step 416, indel mutations are identified by comparing the sequence information to a reference sequence. For example, if the tissue sample is a human tissue sample, such as a tumor sample taken from a human patient, the reference sequence used in step 416 can be a human reference genome sequence. Sequences can be compared using any suitable alignment algorithm and differences between the sequenced amplicons and the reference sequence can be analyzed to identify insertion and deletion mutations.

In step 418, the portion of insertion and deletion mutations identified in step 416 and repaired by NHEJ is determined. As has been described, the type of non-HR repair used to repair a DSB varies according to the length of microhomology between DNA ends resulting from a DSB. The present disclosure uses this relationship to predict repair method based on the length of microhomology regions flanking indels identified in step 416 (Chang, H., Pannunzio, N., Adachi, N. et al. Non-homologous DNA end joining and alternative pathways to double-strand break repair. Nat Rev Mol Cell Biol 18, 495-506 (2017). https://doi.org/10.1038/nrm.2017.48). The data presented by Chang et al. suggests that NHEJ is the most common method of repairing double strand breaks where the ends of the DSB have perfect microhomology of less than three base pairs. As such, a threshold value for flanking homology of indel sites of, e.g., three base pairs or a value less than three base pairs can be used to predict indels that have been repaired by NHEJ. Further, the overall length of insertion and deletion mutations can be used to determine whether an insertion or deletion was repaired by NHEJ. Insertions of three or fewer nucleotides and deletions of three or fewer nucleotides, regardless of the size of surrounding microhomology, evidence the use of NHEJ to repair a DSB. However, indels where flanking homology has a length that exceeds the total length of the underlying indel mutation are often not repaired by NHEJ and can be excluded from categorization as repaired by NHEJ.

To determine the portion of indels identified in step 416 that were repaired by NHEJ, the microhomology of regions flanking each indel is analyzed and, for each indel loci, the length of the flanking microhomology region can be compared to the threshold value. Indels having flanking microhomology regions greater than the threshold value are categorized as repaired by pathway other than NHEJ (e.g., an alternative end joining pathway). Indels having flanking microhomology regions less than the threshold value are categorized as repaired by NHEJ. While the threshold value is generally described herein as categorizing flanking homology that is “greater than” or “less than” the threshold, the terms “greater than” and “less than” can refer respectively to “greater than or equal to” or “less than or equal to” operators. The portion of indels identified in step 416 can be expressed as a whole number (i.e., a number of indels repaired by NHEJ) and/or as a ratio of indels repaired by NHEJ to all indels identified in step 416. The ratio can be, e.g., a fraction, decimal, percentage, etc. Further, the length of the insertion or deletion mutation can also be compared to a threshold value and insertion or deletion mutations that are smaller than an appropriate threshold can be categorized as repaired by NHEJ even where the flanking homology regions exceed the flanking homology threshold, so long as the size of the flanking homology does not exceed the overall nucleotide length of the insertion or deletion mutation.

In some examples, insertion and deletion mutations can be separately categorized in step 418 and, subsequent to categorization, can be re-aggregated to create a combined portion of indels repaired by NHEJ value. This type of workflow allows separate threshold values to be used to categorize both insertion and deletion mutations. For example, a flanking homology threshold of two nucleotides or fewer and a mutation length threshold of three nucleotides or fewer can be used to categorize insertion mutations, such that any insertion mutation having flanking homology of two nucleotides or fewer or an overall insertion length of three nucleotides or fewer can be categorized as repaired by NHEJ. In these examples, a separate flanking homology threshold of three nucleotides or fewer and a separate mutation length threshold of three nucleotides or fewer can be used to categorize deletion mutations such that any deletion mutation having flanking homology of three nucleotides or fewer or an overall deletion length of three nucleotides or fewer can be categorized as repaired by NHEJ. In examples where separate threshold are used for insertion and deletion mutations, the length of flanking homology can be compared to the length of the mutation for both insertion and deletion mutations to exclude indels where the size of the flanking homology regions exceeds the total length of the underlying indel mutation.

In step 420, an HRD determination is made based on the portion of DSBs repaired by NHEJ generated in step 414. Similar to step 126 of method 100, the threshold value can be determined by correlating the portion of DSB repair performed by NHEJ to GIS. Steps 402-414 (including any relevant substeps) can be performed for cell lines for which GIS is known or, alternatively, can be performed for cell lines for which GIS can be calculated. GIS can be calculated using an existing technique, such as the LOH+LST+TAI described by Patel et al. A correlative scale can then be created to generate GIS information from portions of DSBs repaired by NHEJ produced according to step 418. A linear regression can be used to generate a correlative equation between GIS and portions of DSBs repaired by NHEJ. A threshold GIS value can then correlated to a corresponding portion of DSBs repaired by NHEJ using the regression model and the resultant threshold for NHEJ utilization can be used in step 420.

In step 422, a treatment suitable for targeting HRD positive tumor cells is administered to a patient from which the tissue sample was obtained in step 402. Step 422 is an optional step of method 400 and, in examples of method 400 including step 422, step 422 is a conditional step that is performed based a determination in step 420 that the tissue sample is HRD positive. Step 422 is substantially similar to step 128 of method 100 (FIG. 1) and the description of step 128 herein is applicable to step 422 of method 400.

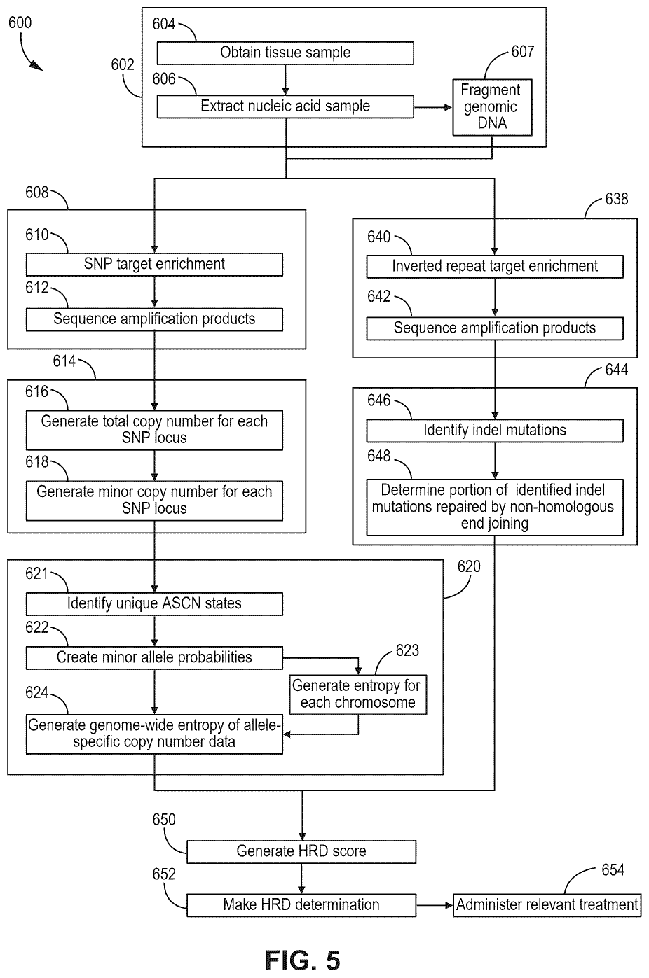

FIG. 5 is a flow diagram of method 600, which combines genome-wide entropy determined according to method 100 with the portion of indel mutations repaired by NHEJ determined according to method 400 to generate a single, combined HRD score for a tissue sample (e.g., a tumor sample). Advantageously and as will be explained in more detail subsequently with respect to the discussion of FIGS. 7, 8, and 9A-9B, the combined HRD score produced by method 600 has a stronger correlation to GIS than genome-wide entropy determined according to method 100 or the portion of indels repaired by NHEJ determined according to method 400 alone.

Method 600 includes steps of preparing a nucleic acid sample (step 602), obtaining a tissue sample (step 604), extracting a nucleic acid sample (step 606), fragmenting genomic DNA (step 607), generating SNP panel data (step 608), amplifying the nucleic acid sample with SNP panel primers (step 610), sequencing amplification products (step 612), generating allele specific copy number data (step 614), generating total copy number information (step 616), generating minor copy number information (step 618), generating an entropy of the allele-specific copy number (ASCN) data (step 620), identifying unique ASCN states (step 621), creating genomic span proportions (step 622), generating entropy for each chromosome (step 623), generating genome-wide entropy of the ASCN data (step 624), generating inverted repeat panel data (step 638), amplifying the nucleic acid sample with indel panel primers (step 640), sequencing amplification products (step 642), determining a portion of indels repaired by NHEJ (step 644), identifying indel mutations based on the sequencing data (step 646), determining a portion of the identified indel mutations repaired by NHEJ (step 648), generating a combined HRD score (step 650), making an HRD determination (step 652), and administering a relevant treatment (step 654). As will be discussed in more detail subsequently, steps 652 and 654 are optional steps of method 600.

Steps 602-607 are substantially similar to steps 102-107 of method 100 (FIG. 1), respectively, and the description of steps 102-107 of method 100 herein is applicable to steps 602-607, respectively, of method 600. Steps 608-624 are substantially similar to steps 108-124 of method 100 (FIG. 1), respectively, and the description of steps 108-124 of method 100 herein is applicable to steps 608-624, respectively, of method 600. Steps 638-648 are substantially similar to steps 408-418 of method 400 (FIG. 4), respectively, and the description of steps 408-418 of method 100 herein is applicable to steps 638-648, respectively, of method 600.

In step 650 of method 600, the genome-wide entropy generated in step 620 (e.g., via steps 621-624) and the portion of DSBs repaired via NHEJ generated in step 644 (e.g., via steps 646-648) are combined into a single HRD score. Numeric values representative of the entropy generated in step 620 and the portion of DSBs repaired via NHEJ can be modified using one or more scalars to create the HRD score. In some examples, a numeric offset can also be applied to adjust the value of the HRD score to, for example, adjust the value of the HRD score to be aligned GIS score for ease of operator use.

In at least some examples, the numeric values (including any scalars and/or offsets applied to the values generated in steps 620 and 644) can be determined by multiple linear regression of the values generated in steps 620 and 644 against GIS. GIS can be characterized for a variety of cell lines and/or samples for which entropy and portion of DSBs repaired by NHEJ can also be generated via method 600. GIS can be generated as described previously with respect to step 126 of method 100 (FIG. 1) and step 420 of method 400 (FIG. 4). A multiple linear regression can be performed to model the relationship between the values generated in steps 620, 644 and GIS. In some of these examples, the scalar and/or offset values created by the multiple linear regression can be adjusted to increase identity between overall HRD score and GIS. Those scalar and offset values can then be used in step 650 to generate the HRD score.

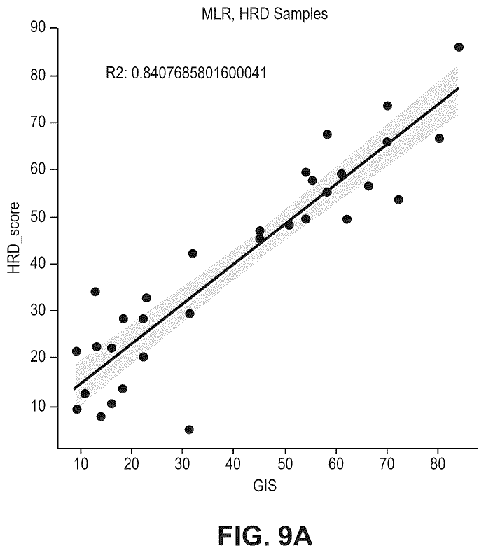

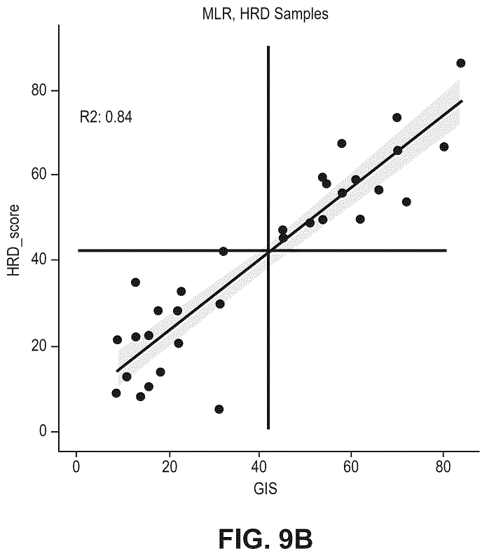

In step 652, HRD status is determined based on the HRD score generated in step 650. HRD status can be determined using a threshold value of HRD score associated with HRD positive and/or HRD negative phenotypes. In examples where a multiple linear regression has been performed to model the relationship between GIS and HRD score, the resultant model can be used to determine a suitable HRD score threshold for determining HRD status by transforming a threshold GIS value into a corresponding HRD score that can be used as a threshold. A specific example of an HRD score threshold derived from a GIS threshold is discussed subsequently and particularly with respect to the discussion of FIG. 9B.

Step 654 is an optional step of method 600 in which a treatment suitable for targeting HRD positive tumor cells is administered to a patient from which the tissue sample was obtained in step 602. Step 654 is an optional step of method 600 and, in examples of method 600 including step 654, step 654 is a conditional step that is performed based a determination in step 652 that the tissue sample is HRD positive. Step 654 is also only performed in examples of method 600 in which the tissue sample is a tumor sample. The treatment administered can be, for example, PARP inhibitor known to be effective in treating tumors deficient in homologous recombination (e.g., BRCA and BRCA-like tumors). PARP inhibitors are only one exemplary class of treatment suitable for treating HRD positive tumors, and other suitable treatments can be used in step 654.

Advantageously, methods 100, 400, and 600 provide three different methods by which HRD status can be evaluated. Method 100 (FIG. 1) provides a measure of HRD status based on information entropy of copy number information and method 400 (FIG. 4) provides a measure of HRD status based on NHEJ utilization. As will be explained subsequently and particularly with reference to FIGS. 7-8, either measure alone can be used to make HRD status determinations.

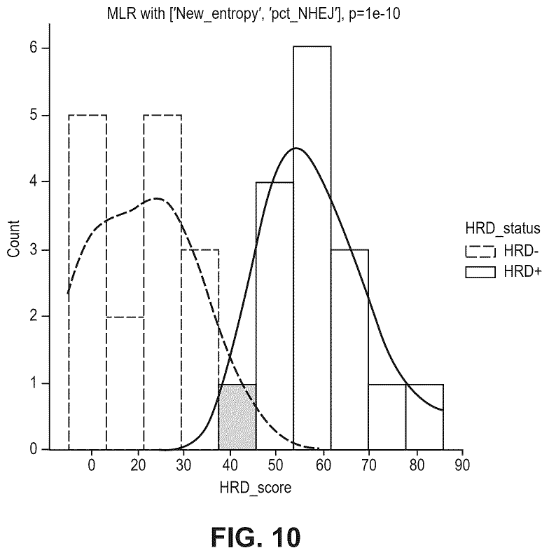

Method 600 (FIG. 5) provides a method of making HRD status determinations made using copy number entropy information and NHEJ utilization by creating a combined HRD score. As will be explained subsequently and particularly with reference to FIGS. 9A-10, the HRD scores produced by method 600 advantageously have improved accuracy for HRD status determination as compared to either underlying measure (i.e., entropy or NHEJ utilization alone).

As described previously, methods 100, 400, and 600 have significantly reduced reagent requirements and sequencing requirements as compared to conventional methods of HRD determination, which largely rely on wide-scale characterization of genomic scarring. Further, as will be described in more detail subsequently with respect to the discussion of FIGS. 7-10, methods 100, 400, and 600 provide accurate methods of HRD status determination while also reducing materials requirements (i.e., by reducing the number of probes and/or primers) as well as reducing the number of required sequencing reads.

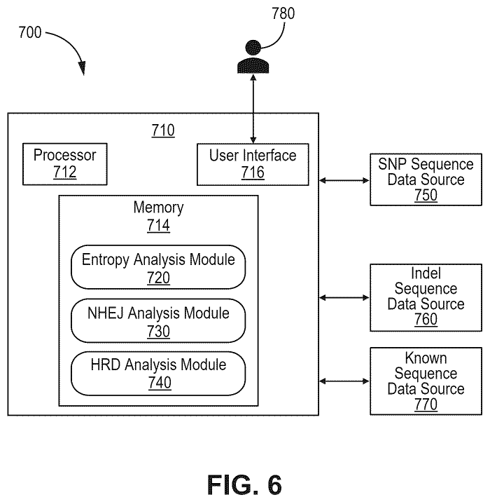

FIG. 6 is a schematic diagram of system 700, which is a system suitable for generating HRD scores and capable of performing relevant steps of method 100 (FIG. 1), method 400 (FIG. 4), and method 600 (FIG. 5). System 700 includes computer 710, SNP sequence data source 750, indel sequence data source 760, and known sequence data source 770. Computer 710 includes processor 712, memory 714, and user interface 716. Memory 714 stores entropy analysis module 720, NHEJ analysis module 730, and HRD analysis module 740.

Processor 712 can execute software, applications, and/or programs stored on memory 714. Examples of processor 712 can include one or more of a processor, a microprocessor, a controller, a digital signal processor (DSP), an application specific integrated circuit (ASIC), a field-programmable gate array (FPGA), or other equivalent discrete or integrated logic circuitry. Processor 712 can be entirely or partially mounted on one or more circuit boards.

Memory 714 is configured to store information and, in some examples, can be described as a computer-readable storage medium. Memory 714, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). In some examples, memory 714 is a temporary memory. As used herein, a temporary memory refers to a memory having a primary purpose that is not long-term storage. Memory 714, in some examples, is described as volatile memory. As used herein, a volatile memory refers to a memory that that the memory does not maintain stored contents when power to the memory 714 is turned off. Examples of volatile memories can include random access memories (RAM), dynamic random access memories (DRAM), static random access memories (SRAM), and other forms of volatile memories. In some examples, the memory is used to store program instructions for execution by the processor. Memory 714, in one example, is used by software or applications running on computer 700 (e.g., by a computer-implemented machine-learning model) to temporarily store information during program execution.

Memory 714, in some examples, also includes one or more computer-readable storage media. The storage media can be configured to store larger amounts of information than volatile memory and, further, can be configured for long-term storage of information. In some examples, memory 714 includes non-volatile storage elements. Examples of such non-volatile storage elements can include, for example, magnetic hard discs, optical discs, floppy discs, flash memories, or forms of electrically programmable memories (EPROM) or electrically erasable and programmable (EEPROM) memories.

User interface 716 is an input and/or output device and/or software interface, and enables an operator (e.g., user 780) to control operation of and/or interact with software elements of computer 710. For example, user interface 716 can be configured to receive inputs from an operator and/or provide outputs. User interface 716 can include one or more of a sound card, a video graphics card, a speaker, a display device (such as a liquid crystal display (LCD), a light emitting diode (LED) display, an organic light emitting diode (OLED) display, etc.), a touchscreen, a keyboard, a mouse, a joystick, or other type of device for facilitating input and/or output of information in a form understandable to users and/or machines.

SNP sequence data source 750 is a data source that stores or otherwise includes SNP data usable with, for example, steps 114-118 of method 100 (FIG. 1) and/or steps 614-618 of method 600 (FIG. 5) to generate ASCN data. Indel sequence data source 760 is a data source that stores or otherwise includes sequence data usable with steps 414-418 of method 400 (FIG. 4) and/or steps 634-638 of method 600 (FIG. 5) to determine a portion of indels repaired via NHEJ. While the data stored to indel sequence data source 760 is generally referred to herein as “indel sequence data,” the sequence data can more broadly include any suitable sequence data for mutation loci at which NHEJ may putatively have occurred. The data of SNP sequence data source 750 can be generated by, for example, steps 102-112 of method 100 (FIG. 1) and/or equivalent steps of method 600 (FIG. 5), and the data of indel sequence data source 760 can be generated by, for example, steps 402-412 of method 400 (FIG. 4) and/or equivalent steps of method 600 (FIG. 5). More broadly, SNP sequence data source 750 and indel sequence data source 760 can include a suitable nucleotide sequencer for generating nucleotide sequence data. The nucleotide sequencer can be any suitable apparatus for generating nucleotide sequence data, including devices for Illumina dye sequencing (Illumina), ion semiconductor sequencing, or any other suitable sequencing technique.

Known sequence data source 770 can be any suitable database or other repository for known sequences that can be used in steps 414-418 of method 400 (FIG. 4) and/or steps 644-648 of method 600 (FIG. 5). Known sequence data source 770 can, in some examples, be a sequence database.

SNP sequence data source 750, indel sequence data source 760, and known sequence data source 770 can also each include one or more databases, memories, etc. for storing sequence data, such as one or more network-connected databases connected to computer 110 via one or more network connections, and, in some examples, can be a network-connected database that is connected to computer 110 via one or more network connections and one or more networks. In some examples, one or more of SNP sequence data source 750, indel sequence data source 760, and known sequence data source 770 can be accessed via the Internet.

Entropy analysis module 720 is a software module of computer 710 and includes one or more programs for calculating entropy based on ASCN data. Entropy analysis module 720 can be configured to perform steps 114-126 of method 100 (FIG. 1) and/or steps 614-624 of method 600 (FIG. 5).

NHEJ analysis module 730 is another software module of computer 710 and includes one or more programs for determining the extent of NHEJ-mediated repair of DSBs. NHEJ analysis module 730 can be configured to perform steps 414-420 of method 400 (FIG. 4) and/or steps 644-648 of method 600 (FIG. 5).

HRD analysis module 740 is yet a further software module of computer 710 and includes one or more programs for determining generating HRD scores according to method 600 and, optionally, making HRD determinations according to step 652 of method 600. NHEJ analysis module 730 can be configured to perform any and/or all of steps 614-624 and 644-652 of method 600 (FIG. 6).

### EXAMPLES

The following examples are illustrative and are not intended to limit the scope of the invention.

### Example 1—HRD Characterization by ASCN Data Entropy

FIG. 7 is a plot of entropy calculated according to method 100 (FIG. 1) against genomic instability score for a cohort of thirty-two samples. More specifically, various FFPE human tumor samples as well as three cell line reference standards of varying HRD status were processed according to step 102 of method 100 to extract genomic DNA. The three cell line reference standards were Seraseq HRD-Negative, Seraseq HRD Low-Positive RM, and Seraseq HRD High-Positive RM standards (Seracare). Ten samples were obtained from Quality in Pathology (QuIP) and were of cells having varying HRD status. The remaining nineteen cell samples were independently obtained.

Target enrichment for each sample was performed (i.e., according to step 110) via AMP (Archer) using a panel of 5,000 primers targeting 5,000 known SNP sites. The resulting amplicons were sequenced to generate SNP panel data describing the abundance of different SNP alleles for each of the 5,000 SNP sites. The SNP panel data was used to generate total copy number of all alleles at each site as well as copy number of the minor allele for each site (i.e., the less abundant allele). The copy number information was used to generate proportions as described with respect to the example of FIG. 3 and further as described with respect to step 122 of method 100. The proportions were used to generate an entropy according to equation 1. In particular, genomic span proportions of unique ASCN states was calculated for each chromosome and, subsequently, entropy was calculated for each chromosome based on those genomic span proportions. Entropy for each sample was then aggregated by summation to produce a single entropy score.

GIS values for the three Seraseq standards were provided by Seracare and, similarly, GIS values were provided by QuIP for the ten samples obtained therefrom. GIS values for the remaining nineteen samples were determined according to the LOH+LST+TAI approach described elsewhere herein. for each sample. Entropy was plotted against GIS for each sample to generate the plot shown in FIG. 7. The trendline shown in FIG. 7 was generated by performing a linear regression of the entropy and GIS data. As shown in FIG. 7, entropy positively correlates with GIS and the regression model has an R-squared of 0.75, indicating that entropy as generated according to method 100 is sufficiently correlative of GIS that entropy can be used to determine HRD status of tissue samples. The regression model shown in FIG. 7 can be used to transform known GIS thresholds for making HRD determinations into ASCN entropies, thereby allowing ASCN entropies to be used to directly make HRD determinations without requiring conversion to GIS.

### Example 2—HRD Characterization by DSB Repair Type

FIG. 8 is a plot of NHEJ repair extent generated according to method 400 (FIG. 4) against genomic instability score for the same cohort of thirty-two samples used to generate the plot of FIG. 7. Target enrichment for each sample was performed (i.e., according to step 410) via AMP (Archer) using a panel of 130 primers targeting 130 known inverted repeat sequences. The resulting amplicons were sequenced to generate indel panel data sequences at each inverted repeat loci. The amplicon sequences were compared by alignment to hg19 to identify insertion and deletion mutations (Genome assembly GRCh37). Indel mutations identified were then examined to determine the length of the insertion and/or deletion as well as the length of surrounding microhomology. Deletions of three nucleotides or fewer or deletion sites having flanking microhomology of three nucleotides or fewer were categorized as repaired by NHEJ. Insertions of three nucleotides or fewer or insertion sites having microhomology of two nucleotides or fewer were also categorized as repaired by NHEJ. The insertions and deletions categorized as repaired by NHEJ were summed and a ratio of indels repaired by NHEJ to all identified indels (i.e., at sequences targeted by the panel of 130 inverted repeat sequences) was created for each sample of the cohort of thirty-two samples. Further, indels where flanking homology exceeded the total length of the indel mutation were excluded from categorization as repaired by NHEJ.

The ratios of indels repaired by NHEJ were converted to percentages representative of the percentage of indels repaired by NHEJ. Those percentages were plotted against GIS (i.e., the GIS values used to generate the plot of FIG. 7) to produce the plot shown in FIG. 8. The trendline in FIG. 8 was generated by performing a linear regression of the entropy and GIS data. As shown in FIG. 8, the percentage of indels repaired by NHEJ was unexpectedly found to negatively correlate with GIS. The regression model has an R-squared of 0.64, indicating that entropy as generated according to method 100 is sufficiently correlative of GIS that entropy can be used to determine HRD status of tissue samples. The regression model shown in FIG. 8 can be used to transform known GIS thresholds for making HRD determinations into threshold percentages of indels repaired by NHEJ, thereby allowing percentage of indels repaired by NHEJ to be used to directly make HRD determinations without requiring conversion to GIS.

As described previously, Example 2 is non-limiting and is merely one method of determining the extent of DSBs repaired by NHEJ. Indels are one example of a mutation that is known be caused by in DSBs, and inverted repeats are merely one example of a genetic feature that is known to result in DSBs. As such, other sequence features can be targeted to understand the extent of DSBs repaired by NHEJ and, further, to make HRD determinations as outlined with respect to Example 2 herein.

### Example 3—HRD Characterization by HRD Score

FIG. 9A is a plot of combined HRD score generated according to method 600 (FIG. 5) against genomic instability score for the same cohort of thirty-two samples used to generate the plot of FIG. 7 and the plot of FIG. 8. The combined HRD scores shown in FIG. 9 were generated from the entropies shown in FIG. 7 and the percentage of NHEJ repair data shown in FIG. 8.

Resultantly, each sample of the cohort of thirty-two samples was processed according to steps 602-648 of method 600 to generate the entropy and percentage NHEJ data used to generate HRD scores. More specifically, each sample was processed according to step 602 of method 600 to extract genomic DNA.

A multiple linear regression was performed to plot a combined HRD score that incorporates both ASCN entropy and percentages of indels repaired by NHEJ against GIS for each cell sample. The GIS data used was the same GIS data used to generate the plots of FIGS. 7-8. The scalars and offsets of the multiple linear regression model were adjusted to improve identity between HRD score and GIS, resulting in the following Equation 2 for generating HRD scores from ASCN entropy and percentages of indels repaired by NHEJ:

$$H R D S = 2.02 {H}^{′} - 0.64 N + 43.95 \qquad [ Equation 2 ]$$

where HRDS is the HRD score, H′ is an entropy generated according to Equation 1, and N is a percentage of indels repaired by NHEJ.

As is shown in FIG. 9A, the multiple linear regression model used to generate Equation 2 has an R-squared value of 0.84, indicating that HRD score according to method 600 can be used to accurately make HRD determinations and, further, that the combined HRD score produced by method 600 has improved correlation to GIS as compared to entropy (FIG. 7; R-squared of 0.75) alone or percentage of indels repaired by NHEJ (FIG. 8; R-squared of 0.64) alone.

FIG. 9B is the plot of FIG. 9A with threshold lines superimposed. The threshold lines shown in FIG. 9B can be used to determine whether a tissue sample is HRD positive. The x-axis threshold is a known threshold that can be used to determine HRD status from GIS. GIS above the threshold indicates that a tissue sample is HRD positive. The y-axis threshold is an HRD score that corresponds (i.e., according to the multiple linear regression model) to the GIS threshold value. As such, the HRD threshold value can also be used to determine whether a sample is HRD positive without requiring conversion of HRD scores generated according to method 600 to GIS. In particular, HRD scores above the HRD score threshold.

A tumor sample taken from a patient that has an HRD score above the HRD threshold shown in FIG. 9B can be classified as HRD positive based on the relationship between the HRD scores disclosed herein and GIS. The HRD positive determination made by HRD score can be used to select a treatment suitable for treating HRD positive tumors, such as PARP inhibitors.

Table 1 below includes GIS values and the HRD scores shown in FIGS. 9A and 9B, as well as the entropies and percentages of indels repaired by NHEJ. Table 1 also includes HRD calls according to the threshold depicted in FIG. 9B. The entropies of Table 1 are the entropies discussed previously with respect to Example 1 and FIG. 7. Similarly, the percentages of indels repaired by NHEJ shown in Table 1 are the percentages of indels repaired by NHEJ discussed previously with respect to Example 2 and FIG. 8. The nineteen independently-obtained samples are indicated in Table 1 by the label “IDT” and a number.

TABLE 1
HRD Score, GIS, Entropy, percentage of indels repaired by NHEJ,
and predicted HRD status for a cohort of thirty-two cell samples

| | | | Predicted | | |
|---|---|---|---|---|---|
| | HRD | | HRD | | % |
| Sample ID | Score | GIS | Status | Entropy | NHEJ |
| Seraseq HRD- | 29.78914 | 31 | HRD− | 14.3865 | 67.74194 |
| Negative | | | | | |
| Seraseq HRD | 59.32121 | 54 | HRD+ | 27.62438 | 63.33333 |
| Low-Positive RM | | | | | |
| Seraseq HRD | 53.76054 | 72 | HRD+ | 27.42703 | 71.42857 |
| High-Positive | | | | | |
| RM | | | | | |
| QuIP #1 | 49.61102 | 54 | HRD+ | 22.55052 | 62.5 |
| QuIP #2 | 56.55444 | 66 | HRD+ | 24.4347 | 57.57576 |
| QuIP #3 | 22.28802 | 13 | HRD− | 10.33062 | 66.66667 |
| QuIP #4 | 66.682 | 80 | HRD+ | 27.05867 | 50 |
| QuIP #5 | 67.67202 | 58 | HRD+ | 22.2837 | 33.33333 |
| QuIP #6 | 49.51428 | 62 | HRD+ | 18.55351 | 50 |
| QuIP #7 | 5.181361 | 31 | HRD− | 1.103493 | 64.28571 |
| QuIP #8 | 86.00286 | 84 | HRD+ | 34.52436 | 43.33333 |
| QuIP #9 | 20.46441 | 22 | HRD− | 13.63953 | 80 |
| QuIP #10 | 10.54996 | 16 | HRD− | 6.804716 | 73.91304 |
| IDT #1 | 12.77565 | 11 | HRD− | 9.128334 | 77.77778 |
| IDT #2 | 7.741485 | 14 | HRD− | 3.124027 | 66.66667 |
| IDT #3 | 13.75596 | 18 | HRD− | 9.343974 | 76.92308 |
| IDT #4 | 34.08489 | 13 | HRD− | 22.19264 | 85.71429 |
| IDT #6 | 9.222392 | 9 | HRD− | 5.362107 | 71.42857 |
| IDT #7 | 21.55693 | 9 | HRD− | 5.154299 | 51.42857 |
| IDT #9 | 45.18617 | 45 | HRD+ | 21.67474 | 66.66667 |
| IDT #11 | 58.85836 | 61 | HRD+ | 30.66519 | 73.68421 |
| IDT #12 | 55.70143 | 58 | HRD+ | 20.18269 | 45.45455 |
| IDT #13 | 28.36394 | 18 | HRD− | 9.203587 | 53.57143 |
| IDT #14 | 73.51119 | 70 | HRD+ | 31.37116 | 52.94118 |
| IDT #15 | 57.74866 | 55 | HRD+ | 21.05331 | 45 |
| IDT #16 | 46.92569 | 45 | HRD+ | 19.52769 | 57.14286 |
| IDT #17 | 22.26485 | 16 | HRD− | 9.26605 | 63.33333 |
| IDT #18 | 65.6943 | 70 | HRD+ | 26.56935 | 50 |
| IDT #19 | 42.46592 | 32 | HRD− | 18.2209 | 60 |
| IDT #20 | 48.61512 | 51 | HRD+ | 21.26731 | 60 |
| IDT #21 | 32.70186 | 23 | HRD− | 15.4898 | 66.66667 |
| IDT #22 | 28.43395 | 22 | HRD− | 12.53294 | 64 |

Further, within the cohort of thirty-two samples, the HRD scores of HRD positive and HRD negative samples were distinguishable and followed a multimodal distribution, with separate modes according to HRD status. FIG. 10 is a combined graph of HRD score abundance (represented as a line) as well as HRD scores grouped into the nearest value of 10 and count number for each grouping (represented as a bar graph). The HRD scores shown in FIG. 10 are the HRD scores shown in FIGS. 9A-9B and also in Table 1. FIG. 10 illustrates that there is a multimodal distribution of HRD score with two separate modes correlated to HRD positive and HRD negative status. Notably, the transition point between the HRD negative and HRD positive modes illustrated in FIG. 10 generally corresponds to the HRD score threshold shown in FIG. 9B. The distribution shown in FIG. 10 further illustrates the ability of HRD scores generated according to method 600 to accurately determine HRD status.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A method of generating a homologous recombination deficiency score, the method comprising:
generating single nucleotide polymorphism (SNP) panel data from a nucleic acid sample, the SNP panel data describing allele abundance at each SNP locus of a plurality of SNP loci;
generating allele specific copy number data for the plurality of SNP loci based on the SNP panel data;
determining an entropy of the allele specific copy number data;
generating double strand break (DSB) feature panel data from the nucleic acid sample, the DSB feature panel data describing nucleotide sequences at a plurality of DSB feature loci, each DSB feature locus of the plurality of DSB feature loci including at least one sequence feature associated with DSBs;
comparing the DSB feature panel data to a known genomic sequence to identify a set of DSB mutations;
determining a portion of the set of DSB mutations that are repaired by non-homologous end joining; and
generating the homologous recombination deficiency score from the entropy of the allele specific copy number data and the portion of the set of DSB mutations repaired by non-homologous end joining.

2. The method of claim 1, and further comprising:
extracting the nucleic acid sample from a tissue sample.

3-4. (canceled)

5. The method of claim 2, and further comprising:
obtaining the tissue sample from a patient;
determining that the homologous recombination deficiency score is above a threshold homologous recombination deficiency score indicative of a homologous repair deficiency phenotype; and
administering a PARP inhibitor to the patient in response to determining that the homologous recombination deficiency score is above the threshold homologous recombination deficiency score.

6. The method of claim 1, and wherein generating the homologous recombination deficiency score comprises:
generating the homologous recombination deficiency score using a multiple linear regression model that correlates homologous recombination deficiency score to genomic instability scores.

7. The method of claim 6, and further comprising:
generating model SNP panel data for a plurality of model nucleic acid samples, the SNP panel data describing, for each model nucleic acid sample of the plurality of model nucleic acid samples, allele abundance at each SNP locus of the plurality of SNP loci;
generating model allele specific copy number data for the plurality of model nucleic acid samples based on the SNP panel data;
determining a plurality of model entropies for the plurality model nucleic acid samples based on the model allele specific copy number data for the plurality of model nucleic acid samples;
generating model DSB feature panel data for the plurality of model nucleic acid samples, the model DSB feature panel data describing, for each model nucleic acid sample of the plurality of model nucleic acid samples, nucleotide sequences at a plurality of inverted repeat loci;
comparing the model DSB feature panel data to the known genomic sequence to identify a plurality of model sets of DSB mutations;
determining, for each model set of DSB mutations of the plurality of model sets of DSB mutations, a model portion that is repaired by non-homologous end joining, thereby determining a plurality of model portions repaired by non-homologous end joining;
generating a plurality of model homologous recombination deficiency scores by, for each model nucleic acid sample, combining a corresponding model entropy of the plurality of model entropies and a corresponding model portion of the plurality of model portions;
retrieving a plurality of model genomic instability scores for the plurality of model nucleic acid samples, each genomic instability score of the plurality of model genomic instability scores descriptive of a different one model nucleic acid sample of the plurality of model nucleic acid samples; and
generating the multiple linear regression model based on the plurality of model homologous recombination deficiency scores and the plurality of model genomic instability scores.

8-10. (canceled)

11. The method of claim 1, wherein generating SNP panel data from the nucleic acid sample comprises:
performing targeted enrichment of the nucleic acid sample to generate enriched SNP fragments, each enriched SNP fragment including an SNP locus of the plurality of SNP loci; and
sequencing the enriched SNP fragments to generate SNP sequencing data describing allele abundance at the plurality of SNP loci.

12-15. (canceled)

16. The method of claim 1, wherein generating DSB feature panel data from the nucleic acid sample comprises:
performing targeted enrichment of the nucleic acid sample to generate enriched DSB feature fragments, each enriched DSB feature fragment including at least one DSB feature locus of the plurality of DSB feature loci;
sequencing the enriched DSB feature-containing fragments to generate DSB feature sequencing data describing nucleotide sequences at the plurality of DSB feature loci.

17-19. (canceled)

20. The method of claim 1, wherein the plurality of DSB feature loci is a plurality of inverted repeat loci.

21. The method of claim 20, wherein generating DSB feature panel data from the nucleic acid sample comprises:
performing target enrichment of the nucleic acid sample using a set of insertion-deletion panel primers to create insertion-deletion panel amplification products; and
sequencing the insertion-deletion panel amplification products to generate insertion-deletion sequencing data describing nucleotide sequences at the plurality of inverted repeat loci.

22. (canceled)

23. The method of claim 1, wherein the set of DSB mutations are a set of insertion-deletion mutations.

24. The method of claim 23, wherein:
comparing the DSB feature panel data to the known genomic sequence to identify the set insertion-deletion mutations comprises analyzing the plurality of nucleotide sequences to identify a plurality of insertion-deletion mutation loci, and
determining the portion of the set of DSB mutations that are repaired by non-homologous end joining comprises determining a portion of the plurality of insertion-deletion mutations repaired by non-homologous end joining.

25. (canceled)

26. The method of claim 24, wherein determining the portion of the set of insertion-deletion mutations that are repaired by non-homologous end joining comprises:
identifying, based on the reference sequence, a microhomology region flanking each insertion-deletion mutation locus of the plurality of insertion-deletion mutation loci, thereby identifying a plurality of microhomology regions;
generating a microhomology length for each microhomology region of the plurality of microhomology regions, thereby determining a plurality of microhomology lengths; and
determining the portion of the set of insertion-deletion mutations that are repaired by non-homologous end joining based on the plurality of microhomology lengths.

27. The method of claim 26, and further comprising:
analyzing the set of insertion-deletion mutations to, for each insertion-deletion loci, generate an insertion-deletion mutation length, thereby generating a plurality of insertion-deletion mutation lengths, and wherein determining the portion of the set of insertion-deletion mutations that are repaired by non-homologous end joining based on the plurality of microhomology lengths comprises determining the portion of the set of insertion-deletion mutations that are repaired by non-homologous end joining based on the plurality of microhomology lengths and the plurality of insertion-deletion mutation lengths.

28. The method of claim 27, wherein each insertion-deletion mutation of the portion of the set of insertion-deletion mutations that are repaired by non-homologous end joining have at least one of a microhomology length less than a first threshold base pair length and an insertion-deletion mutation length than a second threshold base pair length.

29. The method of claim 27, wherein:
analyzing the set of insertion-deletion mutations further comprises analyzing the set of insertion-deletion mutations to identify a set of insertion mutations and a set of deletion mutations, and
generating the portion of the set of insertion-deletion mutations that are repaired by non-homologous end joining comprises:
generating a portion of the set of insertion mutations that are repaired by non-homologous end joining;
generating a portion of the set of deletion mutations that are repaired by non-homologous end joining; and
combining the portion of the set of insertion mutations that are repaired by non-homologous end joining and portion of the set of deletion mutations that are repaired by non-homologous end joining to generate the portion of the set insertion-deletion mutations that are repaired by non-homologous end joining.

30. (canceled)

31. The method of claim 29, wherein:
each insertion mutation of the portion of the set of insertion mutations that are repaired by non-homologous end joining have at least one of a microhomology length less than a first threshold base pair length and an insertion-deletion mutation length than a second threshold base pair length, and
each deletion mutation of the portion of the set of deletion mutations that are repaired by non-homologous end joining have at least one of a microhomology length less than a third threshold base pair length and an insertion-deletion mutation length than a fourth threshold base pair length.

32. (canceled)

33. The method of claim 31, wherein:
the first threshold base pair length is two nucleotides,
the second threshold base pair length is three nucleotides,
the third threshold base pair length is three nucleotides, and
the fourth threshold base pair length is three nucleotides.

34-35. (canceled)

36. The method of claim 1, wherein generating allele specific copy number data for the plurality of SNP loci based on the SNP panel data comprises generating, for each SNP loci, a total copy number value for all alleles of the SNP loci and a minor copy number value for a minor allele of the SNP loci, thereby generating a plurality of total copy number values and a plurality of minor copy number values.

37. The method of claim 36, wherein determining the entropy of the allele specific copy number data comprises:
identifying a plurality of unique allele specific copy number states from the allele specific copy number data, each unique allele specific copy number state having a different combination of minor copy number value and total copy number value;
determining a genomic span for each unique allele specific copy number state, thereby generating a plurality of genomic spans;
determining a total genomic span for all unique allele specific copy number states;
generating, for each genomic span of the plurality of genomic spans, a genomic span proportion based on the respective genomic span and the total genomic span, thereby generating a plurality of genomic span proportions; and
determining the entropy based on the plurality of genomic span proportions.

38. The method of claim 37, wherein determining entropy based on the plurality of genomic span proportions comprises determining entropy according to the following equation:
$${H}^{′} = - {\sum }_{i = 1}^{R} {p}_{i} {log}_{2} {p}_{i}$$
wherein:
H′ is entropy;
pᵢ is a single genomic span proportion of the plurality of genomic span proportions; and
R is a numerosity of the plurality of the genomic span proportions.

39-137. (canceled)
